(19) United States
(12) Patent Application Publication
(10) Pub. No.: US 2018/0208566 A1
(43) Pub. Date: Jul. 26, 2018

(54) Deuterated Meclizine

(71) Applicant: CONCERT Pharmaceuticals, Inc., Lexington, MA (US)

(72) Inventors: Philip B. Graham, Lexington, MA (US); Julie F. Liu, Lexington, MA (US)

(21) Appl. No.: 15/745,929

(22) PCT Filed: Jul. 21, 2016
(86) PCT No.: PCT/US16/43364

§ 371 Date: Jan. 18, 2018

Related U.S. Application Data
(60) Provisional application No. 62/195,162, filed on Jul. 21, 2015.

(51) Int. Cl. C07D 295/073; A61K 45/06; A61K 31/495

(52) U.S. Cl. CPC C07D 295/073; A61K 31/495; A61K 45/06

(57) ABSTRACT

This invention relates to deuterated forms of meclizine, and pharmaceutically acceptable salts or hydrates thereof. This invention also provides compositions comprising a compound of this invention and the use of such compositions in methods of treating diseases and conditions that are beneficially treated by administering meclizine and other Constitutive Androstane Receptor agonists or FGFR3 antagonists.

### RELATED APPLICATION(S)

This application claims the benefit of U.S. Provisional Application No. 62/195,162, filed on Jul. 21, 2015. The entire teachings of the above application(s) are incorporated herein by reference.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

### SUMMARY OF THE INVENTION

This invention relates to deuterated forms of meclizine, and pharmaceutically acceptable salts and hydrates thereof. In one aspect, the invention provides a compound of Formula I:

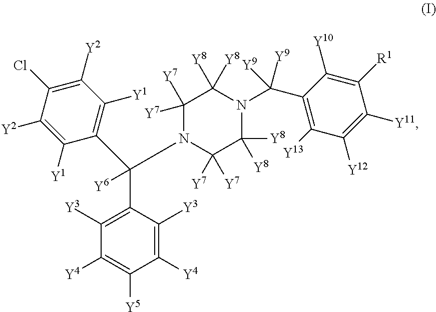

or a pharmaceutically acceptable salt or hydrate thereof, wherein each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium; R¹ is —CH₃, —CH₂D, —CHD₂, or —CD₃; and when each Y is hydrogen, then R¹ is —CHD, —CHD₂, or —CD₃. In some embodiments, in the compound of Formula I, when each Y⁷ and each Y⁸ is deuterium and R¹ is —CH₃, at least one of Y¹, Y², Y³, Y⁵, Y⁶, Y⁹, Y¹⁰, Y¹¹, Y¹² or Y¹³ is deuterium.

This invention also provides compositions comprising a compound of this invention, including pharmaceutical compositions comprising a compound of this invention and a pharmaceutically acceptable carrier. This invention also provides the use of such compounds and compositions in methods of treating diseases and conditions that are beneficially treated by administering meclizine and other Constitutive Androstane Receptor agonists. Some exemplary embodiments include a method of treating a disease or condition selected from Huntington's disease and other polyQ disorders; ischemia-perfusion injury; heart attack; stroke and other diseases involving oxidative damage; achondroplasia, cartilage hypoplasia, Tana Tofo Rick bone dysplasia, Crouzon's disease, distal middle limb dysplasia, Mu severe cartilage with developmental delay, acanthosis nigricans and other systemic bone diseases characterized by over-activation of FGFR3; smoking/nicotine addiction, and vertigo, the method comprising the step of administering to a subject in need thereof a pharmaceutically acceptable composition of the present invention.

### DETAILED DESCRIPTION OF THE INVENTION

Meclizine, also known as 1-[(4-chlorophenyl)phenylmethyl]-4-[(3-methylphenyl)methyl]piperazine, is a Constitutive Androstane Receptor (CAR) agonist that was approved over a half century ago for the treatment of nausea and vomiting. Meclizine has been reported to possess anti-histamine, anti-muscarinic and anti-oxidative phosphorylation properties. This has led to animal studies suggesting that meclizine would be useful as a neuroprotective agent in Huntington's disease and other polyQ toxicity disorders (Gohil, V M et al., Human Mol Gen 20(2), pp. 294-300 (2011); as a cardioprotective and neuroprotective agent in ischemia-perfusion injury and as a potential prophylactic against heart attack, stroke and other diseases involving oxidative damage (Gohil, V M et al., Nat Biotechnol, 28(3), pp. 249-55 (2010); as a treatment for achondroplasia (Matsushita, M et al., Endocrinology, 156(2), pp. 548-54 (2015)) and other systemic bone diseases characterized by over-activation of FGFR3, such as cartilage hypoplasia, Tana Tofo Rick bone dysplasia, Crouzon's disease, distal middle limb dysplasia, Mu severe cartilage with developmental delay and acanthosis nigricans (PCT publication WO2013131847); as a treatment for smoking cessation; and as treatment for vertigo.

Meclizine is in phase II human clinical trials for smoking cessation. Meclizine is also being compared to dimenhydrinate for tolerability and efficacy in a phase III clinical trial for acute vertigo. A phase I study is evaluating the safety and efficacy of meclizine on pre-pulse inhibition.

Despite the beneficial activities of meclizine, there is a continuing need for additional compounds to treat the aforementioned diseases and conditions.

### Definitions

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of Compound 1 will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In one embodiment, the acids commonly employed to form pharmaceutically acceptable salts include the above-listed inorganic acids, wherein at least one hydrogen is replaced with deuterium.

In certain embodiments the hydrates of the compounds of Formula I, Ia or Ib, are monohydrates or dihydrates.

In certain embodiments, the compounds of the invention are in the form of a hydrochloride salt. In one aspect of these embodiments, the compound is a dihydrochloride monohydrate.

The compounds of the present invention (e.g., compounds of Formula I, Ia or Ib), may contain one or more asymmetric carbon atoms, for example, as the result of deuterium substitution or otherwise. As such, compounds of this invention can exist as either individual enantiomers, or mixtures of two or more enantiomers. Accordingly, a compound of the present invention may exist as either a racemic mixture or a scalemic mixture, or as individual respective stereoisomers that are substantially free from another possible stereoisomer. “Stereoisomer” refers to both enantiomers and diastereomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Tert” and “t-” each refer to tertiary. “US” refers to the United States of America.

“Substituted with deuterium” refers to the replacement of one or more hydrogen atoms with a corresponding number of deuterium atoms.

Throughout this specification, a variable may be referred to generally (e.g.,“each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

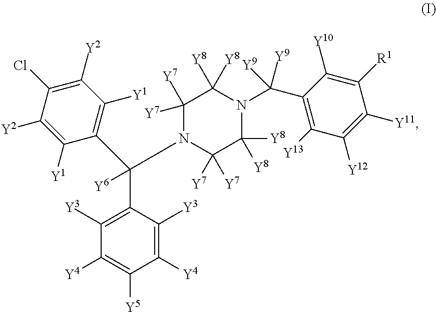

or a pharmaceutically acceptable salt or hydrate thereof, wherein:

each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium;

R¹ is —CH₃, —CH₂D, —CHD₂, or —CD₃; and

when each Y is hydrogen, then R¹ is —CH₂D, —CHD₂, or —CD₃.

In some embodiments, the compound of Formula I is a compound of Formula Ia:

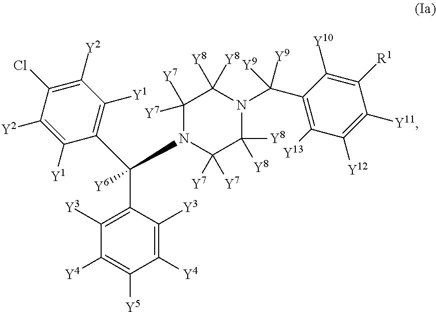

or a pharmaceutically acceptable salt or hydrate thereof, wherein:

each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium;

R¹ is —CH₃, —CH₂D, —CHD₂, or —CD₃; and

when each Y is hydrogen, then R¹ is —CH₂D, —CHD₂, or —CD₃.

In some embodiments, the compound of Formula I is a compound of Formula Ib:

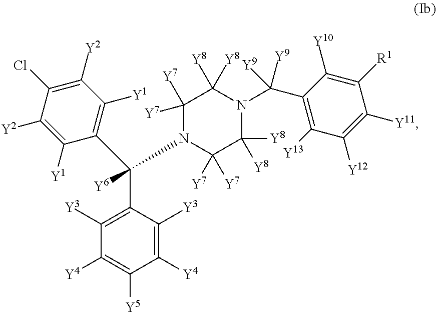

or a pharmaceutically acceptable salt or hydrate thereof, wherein:

each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium;

R¹ is —CH₃, —CH₂D, —CHD₂, or ^{—}CD₃;

when each Y is hydrogen, then R¹ is —CH₂D, —CHD₂, or —CD₃.

In some embodiments of Formula I, Ia or Ib, when each Y⁷ and each Y⁸ is deuterium and R¹ is —CH₃, at least one of Y¹, Y², Y³, Y⁴, Y⁵, Y⁶, Y⁹, Y¹⁰, Y¹¹, Y¹² or Y¹³ is deuterium.

In some embodiments of Formula I, Ia or Ib, each Y¹ is the same; and each Y² is the same. In one aspect of these embodiments, each Y¹ and each Y² are all the same. In one aspect of these embodiments, each Y¹ and each Y² is hydrogen. In an alternate aspect of these embodiments, each Y¹ and each Y² is deuterium.

In some embodiments of Formula I, Ia or Ib, each Y³ is the same; and each Y⁴ is the same. In one aspect of these embodiments, each Y³, each Y⁴ and Y⁵ are all the same. In one aspect of these embodiments, each Y³, each Y⁴, and Y⁵ is hydrogen. In an alternate aspect of these embodiments, each Y³, each Y⁴, and Y⁵ is deuterium.

In some embodiments of Formula I, Ia or Ib, each Y⁷ is the same; and each Y⁸ is the same. In one aspect of these embodiments, each Y⁷ and each Y⁸ is hydrogen. In an alternate aspect of these embodiments, each Y⁷ and each Y⁸ is deuterium. In one aspect of these embodiments, each Y⁷ is deuterium and each Y⁸ is hydrogen. In an alternate aspect of these embodiments, each Y⁷ is hydrogen and each Y⁸ is deuterium.

In some embodiments of Formula I, Ia or Ib, each Y⁹ is the same. In one aspect of these embodiments, each Y⁹ is hydrogen. In an alternate aspect of these embodiments, each Y⁹ is deuterium.

In some embodiments of Formula I, Ia or Ib, Y¹⁰, Y¹¹, Y¹² and Y¹³ are the same. In one aspect of these embodiments, each of Y¹⁰, Y¹¹, Y¹² and Y¹³ is hydrogen. In an alternate aspect of these embodiments, each of Y¹⁰, Y¹¹, Y¹² and Y¹³ is deuterium.

In some embodiments of Formula I, Ia or Ib, R¹ is selected from —CH₃ and —CD₃. In one aspect of these embodiments, R¹ is —CH₃. In an alternate aspect of these embodiments, R¹ is —CD₃.

In some embodiments of Formula I, Ia or Ib, each Y¹ and each Y² are all the same; each Y³, each Y⁴ and Y⁵ are all the same; each Y⁹ is the same; Y¹⁰, Y¹¹, Y¹² and Y¹³ are the same; and R¹ is selected from —CH₃ and —CD₃. In one aspect of these embodiments, each Y⁷ is the same; and each Y⁸ is the same. In a more specific aspect of these embodiments, each Y⁷ and each Y⁸ are all the same. In an even more specific aspect of these embodiments, each Y⁷ and each Y⁸ are deuterium. In another even more specific aspect of these embodiments, each Y⁷ and each Y⁸ are hydrogen.

In one embodiment of Formula I, each Y¹ and each Y² are all the same; each Y³, each Y⁴ and Y⁵ are all the same; each Y⁷ and each Y⁸ are all the same; Y¹⁰, Y¹¹, Y¹² and Y¹³ are the same; and the compound is selected from any one of the compounds set forth in Table 1 (below):

TABLE 1
Exemplary Embodiments of Formula I

| | | | | | | Y¹⁰/Y¹¹/ | |
|---|---|---|---|---|---|---|---|
| Compound # | Y¹/Y² | Y³/Y⁴/Y⁵ | Y⁶ | Y⁷/Y⁸ | Y⁹ | Y¹²/Y¹³ | R¹ |
| 100 | D | H | H | H | H | H | CH₃ |
| 101 | H | D | H | H | H | H | CH₃ |
| 102 | H | H | D | H | H | H | CH₃ |
| 103 | H | H | H | H | D | H | CH₃ |
| 104 | H | H | H | H | H | D | CH₃ |
| 105 | D | D | H | H | H | H | CH₃ |
| 106 | D | H | D | H | H | H | CH₃ |
| 107 | D | H | H | D | H | H | CH₃ |
| 108 | D | H | H | H | D | H | CH₃ |
| 109 | D | H | H | H | H | D | CH₃ |
| 110 | H | D | D | H | H | H | CH₃ |
| 111 | H | D | H | D | H | H | CH₃ |
| 112 | H | D | H | H | D | H | CH₃ |
| 113 | H | D | H | H | H | D | CH₃ |
| 114 | H | H | D | D | H | H | CH₃ |
| 115 | H | H | D | H | D | H | CH₃ |
| 116 | H | H | D | H | H | D | CH₃ |
| 117 | H | H | H | D | D | H | CH₃ |
| 118 | H | H | H | D | H | D | CH₃ |
| 119 | H | H | H | H | D | D | CH₃ |
| 120 | D | D | D | H | H | H | CH₃ |
| 121 | D | D | H | D | H | H | CH₃ |
| 122 | D | D | H | H | D | H | CH₃ |
| 123 | D | D | H | H | H | D | CH₃ |
| 124 | D | H | D | D | H | H | CH₃ |
| 125 | D | H | D | H | D | H | CH₃ |
| 126 | D | H | D | H | H | D | CH₃ |
| 127 | D | H | H | D | D | H | CH₃ |
| 128 | D | H | H | D | H | D | CH₃ |
| 129 | D | H | H | H | D | D | CH₃ |
| 130 | H | D | D | D | H | H | CH₃ |
| 131 | H | D | D | H | D | H | CH₃ |
| 132 | H | D | D | H | H | D | CH₃ |
| 133 | H | D | H | D | D | H | CH₃ |
| 134 | H | D | H | D | H | D | CH₃ |
| 135 | H | D | H | H | D | D | CH₃ |
| 136 | H | H | D | D | D | H | CH₃ |
| 137 | H | H | D | D | H | D | CH₃ |
| 138 | H | H | D | H | D | D | CH₃ |
| 139 | H | H | H | D | D | D | CH₃ |
| 140 | D | D | D | D | H | H | CH₃ |
| 141 | D | D | D | H | D | H | CH₃ |
| 142 | D | D | D | H | H | D | CH₃ |
| 143 | D | D | H | D | D | H | CH₃ |
| 144 | D | D | H | D | H | D | CH₃ |
| 145 | D | D | H | H | D | D | CH₃ |
| 146 | D | H | D | D | D | H | CH₃ |
| 147 | D | H | D | D | H | D | CH₃ |
| 148 | D | H | D | H | D | D | CH₃ |
| 149 | D | H | H | D | D | D | CH₃ |
| 150 | H | D | D | D | D | H | CH₃ |
| 151 | H | D | D | D | H | D | CH₃ |
| 152 | H | D | D | H | D | D | CH₃ |
| 153 | H | D | H | D | D | D | CH₃ |
| 154 | H | H | D | D | D | D | CH₃ |
| 155 | D | D | D | D | D | H | CH₃ |
| 156 | D | D | D | D | H | D | CH₃ |
| 157 | D | D | D | H | D | D | CH₃ |
| 158 | D | D | H | D | D | D | CH₃ |
| 159 | D | H | D | D | D | D | CH₃ |
| 160 | H | D | D | D | D | D | CH₃ |
| 161 | D | D | D | D | D | D | CH₃ |
| 162 | H | H | H | H | H | H | CD₃ |
| 163 | D | H | H | H | H | H | CD₃ |
| 164 | H | D | H | H | H | H | CD₃ |
| 165 | H | H | D | H | H | H | CD₃ |
| 166 | H | H | H | D | H | H | CD₃ |
| 167 | H | H | H | H | D | H | CD₃ |
| 168 | H | H | H | H | H | D | CD₃ |
| 169 | D | D | H | H | H | H | CD₃ |
| 170 | D | H | D | H | H | H | CD₃ |
| 171 | D | H | H | D | H | H | CD₃ |
| 172 | D | H | H | H | D | H | CD₃ |
| 173 | D | H | H | H | H | D | CD₃ |
| 174 | H | D | D | H | H | H | CD₃ |
| 175 | H | D | H | D | H | H | CD₃ |
| 176 | H | D | H | H | D | H | CD₃ |
| 177 | H | D | H | H | H | D | CD₃ |
| 178 | H | H | D | D | H | H | CD₃ |
| 179 | H | H | D | H | D | H | CD₃ |
| 180 | H | H | D | H | H | D | CD₃ |
| 181 | H | H | H | D | D | H | CD₃ |
| 182 | H | H | H | D | H | D | CD₃ |
| 183 | H | H | H | H | D | D | CD₃ |
| 184 | D | D | D | H | H | H | CD₃ |
| 185 | D | D | H | D | H | H | CD₃ |
| 186 | D | D | H | H | D | H | CD₃ |
| 187 | D | D | H | H | H | D | CD₃ |
| 188 | D | H | D | D | H | H | CD₃ |
| 189 | D | H | D | H | D | H | CD₃ |
| 190 | D | H | D | H | H | D | CD₃ |
| 191 | D | H | H | D | D | H | CD₃ |
| 192 | D | H | H | D | H | D | CD₃ |
| 193 | D | H | H | H | D | D | CD₃ |
| 194 | H | D | D | D | H | H | CD₃ |
| 195 | H | D | D | H | D | H | CD₃ |
| 196 | H | D | D | H | H | D | CD₃ |
| 197 | H | D | H | D | D | H | CD₃ |
| 198 | H | D | H | D | H | D | CD₃ |
| 199 | H | D | H | H | D | D | CD₃ |
| 200 | H | H | D | D | D | H | CD₃ |
| 201 | H | H | D | D | H | D | CD₃ |
| 202 | H | H | D | H | D | D | CD₃ |
| 203 | H | H | H | D | D | D | CD₃ |
| 204 | D | D | D | D | H | H | CD₃ |
| 205 | D | D | D | H | D | H | CD₃ |
| 206 | D | D | D | H | H | D | CD₃ |
| 207 | D | D | H | D | D | H | CD₃ |
| 208 | D | D | H | D | H | D | CD₃ |
| 209 | D | D | H | H | D | D | CD₃ |
| 210 | D | H | D | D | D | H | CD₃ |
| 211 | D | H | D | D | H | D | CD₃ |
| 212 | D | H | D | H | D | D | CD₃ |
| 213 | D | H | H | D | D | D | CD₃ |
| 214 | H | D | D | D | D | H | CD₃ |
| 215 | H | D | D | D | H | D | CD₃ |
| 216 | H | D | D | H | D | D | CD₃ |
| 217 | H | D | H | D | D | D | CD₃ |
| 218 | H | H | D | D | D | D | CD₃ |
| 219 | D | D | D | D | D | H | CD₃ |
| 220 | D | D | D | D | H | D | CD₃ |
| 221 | D | D | D | H | D | D | CD₃ |
| 222 | D | D | H | D | D | D | CD₃ |
| 223 | D | H | D | D | D | D | CD₃ |
| 224 | H | D | D | D | D | D | CD₃ |
| 225 | D | D | D | D | D | D | CD₃ |

or a pharmaceutically acceptable salt or hydrate thereof, wherein any atom not designated as deuterium is present at its natural isotopic abundance.

In one embodiment of Formula Ia, each Y¹ and each Y² are all the same; each Y³, each Y⁴ and Y⁵ are all the same; each Y⁷ and each Y⁸ are all the same; Y¹⁰, Y¹¹, Y¹² and Y¹³ are the same; and the compound is selected from any one of the compounds set forth in Table 2 (below):

TABLE 2
Exemplary Embodiments of Formula Ia

| | | | | | | Y¹⁰/Y¹¹/ | |
|---|---|---|---|---|---|---|---|
| Compound # | Y¹/Y² | Y³/Y⁴/Y⁵ | Y⁶ | Y⁷/Y⁸ | Y⁹ | Y¹²/Y¹³ | R¹ |
| 100a | D | H | H | H | H | H | CH₃ |
| 101a | H | D | H | H | H | H | CH₃ |
| 102a | H | H | D | H | H | H | CH₃ |
| 103a | H | H | H | H | D | H | CH₃ |
| 104a | H | H | H | H | H | D | CH₃ |
| 105a | D | D | H | H | H | H | CH₃ |
| 106a | D | H | D | H | H | H | CH₃ |
| 107a | D | H | H | D | H | H | CH₃ |
| 108a | D | H | H | H | D | H | CH₃ |
| 109a | D | H | H | H | H | D | CH₃ |
| 110a | H | D | D | H | H | H | CH₃ |
| 111a | H | D | H | D | H | H | CH₃ |
| 112a | H | D | H | H | D | H | CH₃ |
| 113a | H | D | H | H | H | D | CH₃ |
| 114a | H | H | D | D | H | H | CH₃ |
| 115a | H | H | D | H | D | H | CH₃ |
| 116a | H | H | D | H | H | D | CH₃ |
| 117a | H | H | H | D | D | H | CH₃ |
| 118a | H | H | H | D | H | D | CH₃ |
| 119a | H | H | H | H | D | D | CH₃ |
| 120a | D | D | D | H | H | H | CH₃ |
| 121a | D | D | H | D | H | H | CH₃ |
| 122a | D | D | H | H | D | H | CH₃ |
| 123a | D | D | H | H | H | D | CH₃ |
| 124a | D | H | D | D | H | H | CH₃ |
| 125a | D | H | D | H | D | H | CH₃ |
| 126a | D | H | D | H | H | D | CH₃ |
| 127a | D | H | H | D | D | H | CH₃ |
| 128a | D | H | H | D | H | D | CH₃ |
| 129a | D | H | H | H | D | D | CH₃ |
| 130a | H | D | D | D | H | H | CH₃ |
| 131a | H | D | D | H | D | H | CH₃ |
| 132a | H | D | D | H | H | D | CH₃ |
| 133a | H | D | H | D | D | H | CH₃ |
| 134a | H | D | H | D | H | D | CH₃ |
| 135a | H | D | H | H | D | D | CH₃ |
| 136a | H | H | D | D | D | H | CH₃ |
| 137a | H | H | D | D | H | D | CH₃ |
| 138a | H | H | D | H | D | D | CH₃ |
| 139a | H | H | H | D | D | D | CH₃ |
| 140a | D | D | D | D | H | H | CH₃ |
| 141a | D | D | D | H | D | H | CH₃ |
| 142a | D | D | D | H | H | D | CH₃ |
| 143a | D | D | H | D | D | H | CH₃ |
| 144a | D | D | H | D | H | D | CH₃ |
| 145a | D | D | H | H | D | D | CH₃ |
| 146a | D | H | D | D | D | H | CH₃ |
| 147a | D | H | D | D | H | D | CH₃ |
| 148a | D | H | D | H | D | D | CH₃ |
| 149a | D | H | H | D | D | D | CH₃ |
| 150a | H | D | D | D | D | H | CH₃ |
| 151a | H | D | D | D | H | D | CH₃ |
| 152a | H | D | D | H | D | D | CH₃ |
| 153a | H | D | H | D | D | D | CH₃ |
| 154a | H | H | D | D | D | D | CH₃ |
| 155a | D | D | D | D | D | H | CH₃ |
| 156a | D | D | D | D | H | D | CH₃ |
| 157a | D | D | D | H | D | D | CH₃ |
| 158a | D | D | H | D | D | D | CH₃ |
| 159a | D | H | D | D | D | D | CH₃ |
| 160a | H | D | D | D | D | D | CH₃ |
| 161a | D | D | D | D | D | D | CH₃ |
| 162a | H | H | H | H | H | H | CD₃ |
| 163a | D | H | H | H | H | H | CD₃ |
| 164a | H | D | H | H | H | H | CD₃ |
| 165a | H | H | D | H | H | H | CD₃ |
| 166a | H | H | H | D | H | H | CD₃ |
| 167a | H | H | H | H | D | H | CD₃ |
| 168a | H | H | H | H | H | D | CD₃ |
| 169a | D | D | H | H | H | H | CD₃ |
| 170a | D | H | D | H | H | H | CD₃ |
| 171a | D | H | H | D | H | H | CD₃ |
| 172a | D | H | H | H | D | H | CD₃ |
| 173a | D | H | H | H | H | D | CD₃ |
| 174a | H | D | D | H | H | H | CD₃ |
| 175a | H | D | H | D | H | H | CD₃ |
| 176a | H | D | H | H | D | H | CD₃ |
| 177a | H | D | H | H | H | D | CD₃ |
| 178a | H | H | D | D | H | H | CD₃ |
| 179a | H | H | D | H | D | H | CD₃ |
| 180a | H | H | D | H | H | D | CD₃ |
| 181a | H | H | H | D | D | H | CD₃ |
| 182a | H | H | H | D | H | D | CD₃ |
| 183a | H | H | H | H | D | D | CD₃ |
| 184a | D | D | D | H | H | H | CD₃ |
| 185a | D | D | H | D | H | H | CD₃ |
| 186a | D | D | H | H | D | H | CD₃ |
| 187a | D | D | H | H | H | D | CD₃ |
| 188a | D | H | D | D | H | H | CD₃ |
| 189a | D | H | D | H | D | H | CD₃ |
| 190a | D | H | D | H | H | D | CD₃ |
| 191a | D | H | H | D | D | H | CD₃ |
| 192a | D | H | H | D | H | D | CD₃ |
| 193a | D | H | H | H | D | D | CD₃ |
| 194a | H | D | D | D | H | H | CD₃ |
| 195a | H | D | D | H | D | H | CD₃ |
| 196a | H | D | D | H | H | D | CD₃ |
| 197a | H | D | H | D | D | H | CD₃ |
| 198a | H | D | H | D | H | D | CD₃ |
| 199a | H | D | H | H | D | D | CD₃ |
| 200a | H | H | D | D | D | H | CD₃ |
| 201a | H | H | D | D | H | D | CD₃ |
| 202a | H | H | D | H | D | D | CD₃ |
| 203a | H | H | H | D | D | D | CD₃ |
| 204a | D | D | D | D | H | H | CD₃ |
| 205a | D | D | D | H | D | H | CD₃ |
| 206a | D | D | D | H | H | D | CD₃ |
| 207a | D | D | H | D | D | H | CD₃ |
| 208a | D | D | H | D | H | D | CD₃ |
| 209a | D | D | H | H | D | D | CD₃ |
| 210a | D | H | D | D | D | H | CD₃ |
| 211a | D | H | D | D | H | D | CD₃ |
| 212a | D | H | D | H | D | D | CD₃ |
| 213a | D | H | H | D | D | D | CD₃ |
| 214a | H | D | D | D | D | H | CD₃ |
| 215a | H | D | D | D | H | D | CD₃ |
| 216a | H | D | D | H | D | D | CD₃ |
| 217a | H | D | H | D | D | D | CD₃ |
| 218a | H | H | D | D | D | D | CD₃ |
| 219a | D | D | D | D | D | H | CD₃ |
| 220a | D | D | D | D | H | D | CD₃ |
| 221a | D | D | D | H | D | D | CD₃ |
| 222a | D | D | H | D | D | D | CD₃ |
| 223a | D | H | D | D | D | D | CD₃ |
| 224a | H | D | D | D | D | D | CD₃ |
| 225a | D | D | D | D | D | D | CD₃ |

or a pharmaceutically acceptable salt or hydrate thereof, wherein any atom not designated as deuterium is present at its natural isotopic abundance.

In one embodiment of Formula Ib, each Y¹ and each Y² are all the same; each Y³, each Y⁴ and Y⁵ are all the same; each Y⁷ and each Y⁸ are all the same; Y¹⁰, Y¹¹, Y¹² and Y¹³ are the same; and the compound is selected from any one of the compounds set forth in Table 3 (below):

TABLE 3
Exemplary Embodiments of Formula Ib

| | | | | | | Y¹⁰/Y¹¹/ | |
|---|---|---|---|---|---|---|---|
| Compound # | Y¹/Y² | Y³/Y⁴/Y⁵ | Y⁶ | Y⁷/Y⁸ | Y⁹ | Y¹²/Y¹³ | R¹ |
| 100b | D | H | H | H | H | H | CH₃ |
| 101b | H | D | H | H | H | H | CH₃ |
| 102b | H | H | D | H | H | H | CH₃ |
| 103b | H | H | H | H | D | H | CH₃ |
| 104b | H | H | H | H | H | D | CH₃ |
| 105b | D | D | H | H | H | H | CH₃ |
| 106b | D | H | D | H | H | H | CH₃ |
| 107b | D | H | H | D | H | H | CH₃ |
| 108b | D | H | H | H | D | H | CH₃ |
| 109b | D | H | H | H | H | D | CH₃ |
| 110b | H | D | D | H | H | H | CH₃ |
| 111b | H | D | H | D | H | H | CH₃ |
| 112b | H | D | H | H | D | H | CH₃ |
| 113b | H | D | H | H | H | D | CH₃ |
| 114b | H | H | D | D | H | H | CH₃ |
| 115b | H | H | D | H | D | H | CH₃ |
| 116b | H | H | D | H | H | D | CH₃ |
| 117b | H | H | H | D | D | H | CH₃ |
| 118b | H | H | H | D | H | D | CH₃ |
| 119b | H | H | H | H | D | D | CH₃ |
| 120b | D | D | D | H | H | H | CH₃ |
| 121b | D | D | H | D | H | H | CH₃ |
| 122b | D | D | H | H | D | H | CH₃ |
| 123b | D | D | H | H | H | D | CH₃ |
| 124b | D | H | D | D | H | H | CH₃ |
| 125b | D | H | D | H | D | H | CH₃ |
| 126b | D | H | D | H | H | D | CH₃ |
| 127b | D | H | H | D | D | H | CH₃ |
| 128b | D | H | H | D | H | D | CH₃ |
| 129b | D | H | H | H | D | D | CH₃ |
| 130b | H | D | D | D | H | H | CH₃ |
| 131b | H | D | D | H | D | H | CH₃ |
| 132b | H | D | D | H | H | D | CH₃ |
| 133b | H | D | H | D | D | H | CH₃ |
| 134b | H | D | H | D | H | D | CH₃ |
| 135b | H | D | H | H | D | D | CH₃ |
| 136b | H | H | D | D | D | H | CH₃ |
| 137b | H | H | D | D | H | D | CH₃ |
| 138b | H | H | D | H | D | D | CH₃ |
| 139b | H | H | H | D | D | D | CH₃ |
| 140b | D | D | D | D | H | H | CH₃ |
| 141b | D | D | D | H | D | H | CH₃ |
| 142b | D | D | D | H | H | D | CH₃ |
| 143b | D | D | H | D | D | H | CH₃ |
| 144b | D | D | H | D | H | D | CH₃ |
| 145b | D | D | H | H | D | D | CH₃ |
| 146b | D | H | D | D | D | H | CH₃ |
| 147b | D | H | D | D | H | D | CH₃ |
| 148b | D | H | D | H | D | D | CH₃ |
| 149b | D | H | H | D | D | D | CH₃ |
| 150b | H | D | D | D | D | H | CH₃ |
| 151b | H | D | D | D | H | D | CH₃ |
| 152b | H | D | D | H | D | D | CH₃ |
| 153b | H | D | H | D | D | D | CH₃ |
| 154b | H | H | D | D | D | D | CH₃ |
| 155b | D | D | D | D | D | H | CH₃ |
| 156b | D | D | D | D | H | D | CH₃ |
| 157b | D | D | D | H | D | D | CH₃ |
| 158b | D | D | H | D | D | D | CH₃ |
| 159b | D | H | D | D | D | D | CH₃ |
| 160b | H | D | D | D | D | D | CH₃ |
| 161b | D | D | D | D | D | D | CH₃ |
| 162b | H | H | H | H | H | H | CD₃ |
| 163b | D | H | H | H | H | H | CD₃ |
| 164b | H | D | H | H | H | H | CD₃ |
| 165b | H | H | D | H | H | H | CD₃ |
| 166b | H | H | H | D | H | H | CD₃ |
| 167b | H | H | H | H | D | H | CD₃ |
| 168b | H | H | H | H | H | D | CD₃ |
| 169b | D | D | H | H | H | H | CD₃ |
| 170b | D | H | D | H | H | H | CD₃ |
| 171b | D | H | H | D | H | H | CD₃ |
| 172b | D | H | H | H | D | H | CD₃ |
| 173b | D | H | H | H | H | D | CD₃ |
| 174b | H | D | D | H | H | H | CD₃ |
| 175b | H | D | H | D | H | H | CD₃ |
| 176b | H | D | H | H | D | H | CD₃ |
| 177b | H | D | H | H | H | D | CD₃ |
| 178b | H | H | D | D | H | H | CD₃ |
| 179b | H | H | D | H | D | H | CD₃ |
| 180b | H | H | D | H | H | D | CD₃ |
| 181b | H | H | H | D | D | H | CD₃ |
| 182b | H | H | H | D | H | D | CD₃ |
| 183b | H | H | H | H | D | D | CD₃ |
| 184b | D | D | D | H | H | H | CD₃ |
| 185b | D | D | H | D | H | H | CD₃ |
| 186b | D | D | H | H | D | H | CD₃ |
| 187b | D | D | H | H | H | D | CD₃ |
| 188b | D | H | D | D | H | H | CD₃ |
| 189b | D | H | D | H | D | H | CD₃ |
| 190b | D | H | D | H | H | D | CD₃ |
| 191b | D | H | H | D | D | H | CD₃ |
| 192b | D | H | H | D | H | D | CD₃ |
| 193b | D | H | H | H | D | D | CD₃ |
| 194b | H | D | D | D | H | H | CD₃ |
| 195b | H | D | D | H | D | H | CD₃ |
| 196b | H | D | D | H | H | D | CD₃ |
| 197b | H | D | H | D | D | H | CD₃ |
| 198b | H | D | H | D | H | D | CD₃ |
| 199b | H | D | H | H | D | D | CD₃ |
| 200b | H | H | D | D | D | H | CD₃ |
| 201b | H | H | D | D | H | D | CD₃ |
| 202b | H | H | D | H | D | D | CD₃ |
| 203b | H | H | H | D | D | D | CD₃ |
| 204b | D | D | D | D | H | H | CD₃ |
| 205b | D | D | D | H | D | H | CD₃ |
| 206b | D | D | D | H | H | D | CD₃ |
| 207b | D | D | H | D | D | H | CD₃ |
| 208b | D | D | H | D | H | D | CD₃ |
| 209b | D | D | H | H | D | D | CD₃ |
| 210b | D | H | D | D | D | H | CD₃ |
| 211b | D | H | D | D | H | D | CD₃ |
| 212b | D | H | D | H | D | D | CD₃ |
| 213b | D | H | H | D | D | D | CD₃ |
| 214b | H | D | D | D | D | H | CD₃ |
| 215b | H | D | D | D | H | D | CD₃ |
| 216b | H | D | D | H | D | D | CD₃ |
| 217b | H | D | H | D | D | D | CD₃ |
| 218b | H | H | D | D | D | D | CD₃ |
| 219b | D | D | D | D | D | H | CD₃ |
| 220b | D | D | D | D | H | D | CD₃ |
| 221b | D | D | D | H | D | D | CD₃ |
| 222b | D | D | H | D | D | D | CD₃ |
| 223b | D | H | D | D | D | D | CD₃ |
| 224b | H | D | D | D | D | D | CD₃ |
| 225b | D | D | D | D | D | D | CD₃ |

or a pharmaceutically acceptable salt or hydrate thereof, wherein any atom not designated as deuterium is present at its natural isotopic abundance.

In some embodiments, in the compound of Formula I, Ia, or Ib, at least one of Y¹, Y², Y³, Y⁴, Y⁵, Y⁶, Y⁷, Y⁸, Y⁹, Y¹⁰, Y¹¹, Y¹², Y¹³, and R¹ comprises hydrogen.

In another set of embodiments, any atom not designated as deuterium in any of the mbodiments set forth above is present at its natural isotopic abundance.

The synthesis of compounds of Formula I, Formula Ia and Formula Ib may be readily achieved by synthetic chemists of ordinary skill by reference to the Exemplary Synthesis and Examples disclosed herein. Relevant procedures analogous to those of use for the preparation of compounds of Formula I and intermediates thereof are disclosed, for instance in U.S. Pat. No. 2,823,233; and Chinese Patent Publication CN103772321.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula I, Formula Ia and Formula Ib is depicted in Scheme 1, below.

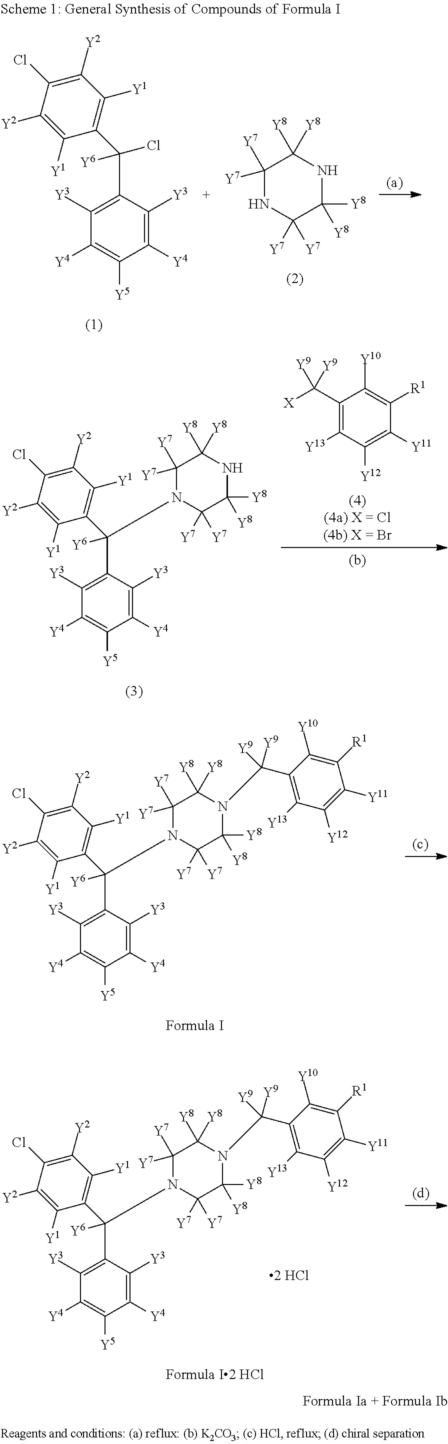

In a manner analogous to a procedure described in CN 103772321, condensation of appropriately deuterated p-halobenzhydryl chloride intermediate (1) with appropriately deuterated piperazine intermediate (2) at elevated temperature furnishes appropriately deuterated benzhydryl-piperazine intermediate (3). Subsequent treatment with appropriately deuterated benzyl halide intermediate (4) in the presence of a base such as potassium carbonate furnishes appropriately deuterated benzyl compounds of Formula I. Finally, treatment of compounds of Formula I with HCl at reflux produces appropriately deuterated.2HCl salt of compounds of Formula I.

It will be appreciated by one skilled in the art that the order of reaction steps may be reversed using synthetic protocols that successfully accomplishes the synthesis of compounds of Formula I. Furthermore, compounds of Formula I contain an asymmetric center, and the procedure shown in scheme 1 produces a racemic mixture. Conventional methods known in the art, such as fractional crystallization or chiral HPLC may be used to resolve the racemic mixture to produce Compounds of Formula Ia and Ib, or for instance, by analogy a procedure described in US 20120136007.

Using commercially available reagents and deuterated reagents that can be readily prepared by known methods, compounds of Formula I, Ia and Ib can be prepared with greater than 90% or greater than 95% deuterium incorporation at each position designated as D (see below for details).

Appropriately deuterated intermediate (1), for use in the preparation of compounds of Formula I according to Scheme 1 may be prepared from corresponding deuterated reagents exemplified in Scheme 2.

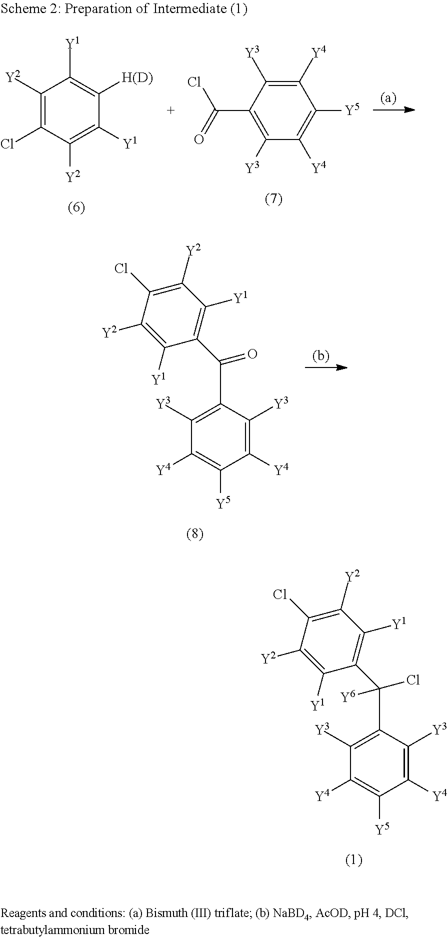

In a manner analogous to a procedure described by Tran, P. et al., Synthetic Communications, 44(20), 2921-2929; 2014, microwave assisted Friedel-Crafts acylation of appropriately deuterated arene intermediate (6) with appropriately deuterated benzoyl chloride intermediate (7) in the presence of bismuth triflate affords appropriately deuterated benzophenone intermediate (8), which is subsequently reduced using reducing agent such as NaBD₄, followed by conversion to the chloride to produce appropriately deuterated benzhydryl chloride intermediate (1), by analogy to a procedure described by Shivaprakash, S. et al., Synthetic Communications, 44(5), 600-609; 2014.

The following intermediates (6) are commercially available: chlorobenzene-d₅ (99 atom % D) (6a) and chlorobenzene-3,5-d₂ (98 atom % D) (6b). Benzene-1,3,5-d₃,2-chloro-(6c) may be prepared in accordance with a procedure described by Makhlynets, O. et al., Chemistry-A European Journal (2010), 16(47), 13995-14006.

Intermediate (7), benzoyl chloride-d₅ (99 atom % D) (7a) is commercially available. When the following commercially available deuterated benzoic acid starting materials are submitted to standard methods known in the art for preparing acid chlorides (for instance using a procedure described by Tang, Q. et al., Journal of the American Chemical Society, 135(12), 4628-4631; 2013), the corresponding appropriately deuterated benzoyl chloride intermediate (7b and 7c) may be obtained: benzoic-2,6-d₂ acid (90-95% atom % D), benzoic-3,5-d₂ acid (98 atom % D).

Using commercially available reagents and deuterated reagents that can be readily prepared by known methods, compounds of Formula I, Ia and Ib can be prepared with greater than 90% or greater than 95% deuterium incorporation at each position designated as D (see below for details).

Appropriately deuterated intermediate (2), for use in the preparation of compounds of Formula I according to Scheme 1 may be prepared from corresponding deuterated reagents exemplified in Scheme 3.

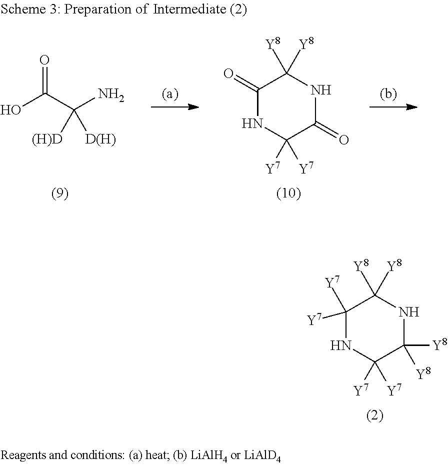

In a manner analogous to a procedure described by Nonappa A. et al., Green Chemistry, 13(5), 1203-1209; 2011, appropriately deuterated glycine intermediate (9) is heated at elevated temperature to furnish appropriately deuterated diketopiperazine intermediate (10), which is subsequently treated with a reducing agent such as LiAlH₄ or LiAlD₄ at elevated temperature to produce appropriately deuterated intermediate (2) by analogy to procedure described in US 20140206673 or WO 2008070619.

Intermediate (9) glycine-2,2-d₂ (98 atom % D) (9a), is commercially available. Furthermore, intermediate (2) piperazine-2,2,3,3,5,5,6,6-d₈ (98 atom % D) (2a), is commercially available.

Using commercially available reagents and deuterated reagents that can be readily prepared by known methods, compounds of Formula I, Ia and Ib can be prepared with greater than 90% or greater than 95% deuterium incorporation at each position designated as D (see below for details).

Appropriately deuterated intermediate (4), for use in the preparation of compounds of Formula I according to Scheme 1 may be prepared from corresponding deuterated reagents exemplified in Scheme 4.

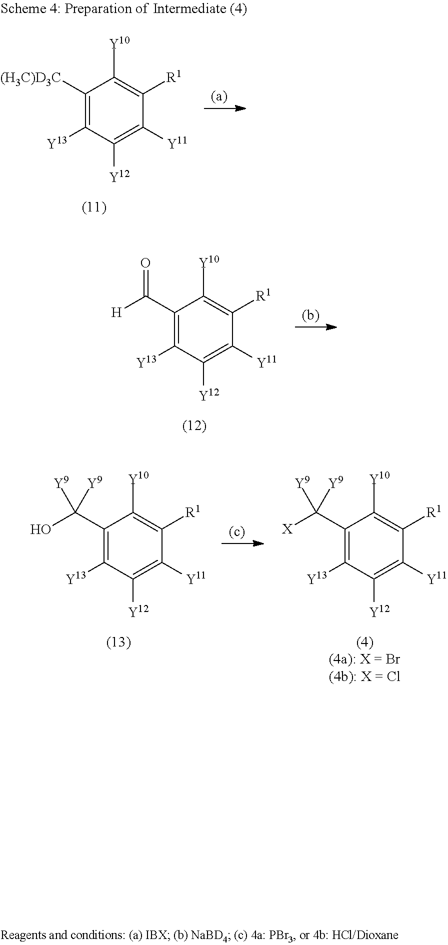

In a manner analogous to a procedure described by Nicolaou, K. et al., Journal of the American Chemical Society, 123(13), 3183-3185; 2001, IBX-mediated oxidation of appropriately deuterated m-xylene intermediate (11) produces appropriately deuterated aryl aldehyde intermediate (12). By analogy to a procedure described by Mao, J. et al., Chemical Communications (Cambridge, United Kingdom) (2014), 50(28), 3692-3694, treatment of intermediate (12) with reducing agent such as NaBD₄ affords appropriately deuterated benzyl alcohol intermediate (13), which is subsequently treated with halogenating agent to produce appropriately deuterated benzyl halide intermediate (4). Halogenation to produce intermediate (4b) is achieved by analogy to a procedure described by Vargas-Rodriguez, Y. et al., Organic Communications, 5(2), 58-63; 2012

The following intermediates (11) are commercially available: m-Xylene-d₁₀ (98 atom % D) (11a), m-Xylene-2,4,5,6-d₄ (98 atom % D) (11b), m-Xylene-(dimethyl-d6) (98 atom % D) (11c).

Benzene, 1-methyl-3-(methyl-d₃) (lid) is prepared in accordance with Kwa, T. et al., Tetrahedron, 25(24), 5771-6; 1969.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I, Ia and Ib and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Pharmaceutical Compositions

The invention also provides pharmaceutical compositions comprising an effective amount of a compound of Formula I, Ia or Ib (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and Unoted Sstates patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). In one aspect of these embodiments, the pharmaceutical composition is formulated for oral administration. In an even more specific aspect of these embodiments, the pharmaceutical composition is a solid dosage form for oral administration.

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, intravesical administration, use of catheters, trocars, projectiles, pluronic gel, stents, sustained drug release polymers or other device which provides for internal access.

In other embodiments, a composition of this invention further comprises a second therapeutic agent. The second therapeutic agent may be selected from any compound or therapeutic agent known to have or that demonstrates advantageous properties when administered with a compound having the same mechanism of action as meclizine. Such agents include those indicated as being useful in combination with meclizine, including but not limited to, those described in PCT publications WO2014/141847, WO2011/150859, WO2011/082426, WO2010/132821, WO2009/151920, WO2009/054007, WO2009/059120 and in U.S. Pat. No. 8,293,749.

Preferably, the second therapeutic agent is an agent useful in the treatment of a disease or condition selected from smoking addiction (e.g., useful in aiding in smoking cessation); vertigo; motion sickness; systemic bone disease; arthritis; nausea; vomiting; neurodegenerative disorders such as ALS, ataxia, Friedrich's dementia, Alzheimer's disease, Parkinson's disease, Huntington's disease; ischemic disorders such as myocardial ischemia, renal ischemia, and stroke. In some embodiments, the second therapeutic agent is an agent useful in the treatment of a disease or condition characterized by mutations leading to increased activity of FGFR3. In some embodiments, the disease or condition characterized by mutations leading to increased activity of FGFR3 is a cancer (e.g., multiple myeloma, urothelial carcinoma, such as bladder cancer, kidney cancer, cancer of the ureter, or cancer of the urethra, prostate cancer, rhabdomycosarcoma, non-small cell lung cancer (NSCLC), specifically NSCLC treated with Epidermal Growth Factor Receptor (EGFR) tyrosine kinase inhibitors, oral squamous cell carcinoma, or thanatophoric dysplasia type II). In other embodiments, then second therapeutic agent is one that has side effects which are attenuated or eliminated by the compounds of the invention. These other therapeutic agents include anti-cancer agents, oral contraceptives, and other agents known to cause nausea and/or vomiting.

In some embodiments, the second therapeutic agent is selected from a nicotine supplement, such as a nicotine patch or nicotine gum. In one aspect of these embodiments the second agent is a nicotine patch.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In some embodiments, an effective amount of a compound of this invention can range from 1-100 mg/dose administered once to five times a day. In one aspect of these embodiments an effective dose is selected from 5-100 mg/dose, 5-50 mg/dose, 5-25 mg/dose, 5-12.5 mg/dose, 10-100 mg/dose, 10-50 mg/dose, 12.5-50 mg/dose, 12.5-25 mg/dose and 25-50 mg/dose, each of the above administered one to three times daily.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for meclizine.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

Certain aspects of the present invention provide a method of agonizing the activity of the Constitutive Androstane Receptor in a cell, comprising contacting the cell with one or more compounds of Formula I, Ia or Ib, or a pharmaceutically acceptable salt thereof.

Certain aspects of the present invention provide a method of agonizing the activity of the human pregnane X receptor (PXR) in a cell, comprising contacting the cell with one or more compounds of Formula I, Ia or Ib, or a pharmaceutically acceptable salt thereof.

Certain aspects of the present invention provide a method of antagonizing or reducing the activity of the Fibroblast Growth Factor Receptor 3 (FGFR3) in a cell, comprising contacting the cell with one or more compounds of Formula I, Ia, or Ib, or a pharmaceutically acceptable salt thereof. In some embodiments, the method antagonizes mutants of FGFR3. In some embodiments, the compound of Formula I, Ia, or Ib is an inverse agonist of constitutively active FGFR3 mutants.

Certain aspects of the present invention provide a method of modulating mitochondrial respiration in a cell, comprising contacting the cell with one or more compounds of Formula I, Ia, or Ib, or a pharmaceutically acceptable salt thereof. In some embodiments, the method modulates mitochondrial respiration by inhibiting oxidative phosphorylation.

Certain aspects of the present invention provide a method of treating a disease that is beneficially treated by meclizine in a subject in need thereof, comprising the step of administering to the subject an effective amount of a compound or a composition of this invention. In one embodiment the subject is a patient in need of such treatment. Such diseases include, but are not limited to Huntington's disease and other polyQ disorders; ischemia-perfusion injury; heart attack; stroke and other diseases involving oxidative damage; achondroplasia, cartilage hypoplasia, Tana Tofo Rick bone dysplasia, Crouzon's disease, distal middle limb dysplasia, Mu severe cartilage with developmental delay, acanthosis nigricans and other systemic bone diseases characterized by over-activation of FGFR3; smoking/nicotine addiction, and vertigo.

In one particular embodiment, the method of this invention is used to aid in smoking cessation or to treat vertigo in a subject in need thereof.

In some embodiments, the method treats a disease or disorder characterized by mutations leading to increased activity of FGFR3. In some embodiments, the method treats a solid or hematological cancer characterized by mutations leading to increased activity of FGFR3. Exemplary cancers include multiple myeloma [see US 2015/0165067], bladder cancer and other urothelial cancers (e.g., cancer of the kidney, ureter, or urethra) [see US 2015/0165067; WO2015109218; Clin Cancer Res Nov 2005 (11) (21) 7709-7719; DOI: 10.1158/1078-0432.CCR-05-1130], prostate cancer [see Molecular Medicine Reports (2015), 11, (2), 1469-1475. Publisher: (Spandidos Publications Ltd.) CODEN:MMROA5 ISSN:1791-299], rhabdomycosarcoma [see British Journal of Cancer (2009), 101, (12), 2030-2037. Publisher: (Nature Publishing Group,) CODEN:BJCAAI ISSN:0007-0920], non-small cell lung cancer (NSCLC), specifically NSCLC treated with Epidermal Growth Factor Receptor (EGFR) tyrosine kinase inhibitors [see PLoS One (2010), 5, (11), e14117. Publisher: (Public Library of Science,) CODEN:POLNCL ISSN:1932-6203], oral squamous cell carcinoma, and thanatophoric dysplasia type II.

In some embodiments, the FGFR3 mutation is a G38OR mutation, a G375C/G364E mutation, a G697C mutation, a K650E mutation, a K650M mutation, a V555M mutation or a S294C/Y375C mutation.

In some embodiments, the disease or disorder characterized by mutations leading to increased activity of FGFR3 is achondroplasia and the FGFR3 mutation is a G38OR mutation.

In some embodiments, the disease or disorder characterized by mutations leading to increased activity of FGFR3 is achondroplasia and the FGFR3 mutation is a G375C/G364E mutation.

In some embodiments, the disease or disorder characterized by mutations leading to increased activity of FGFR3 is an oral squamous cell carcinoma and the FGFR3 mutation is a G697C mutation.

In some embodiments, the disease or disorder characterized by mutations leading to increased activity of FGFR3 is thanatophoric dysplasia type II or multiple myeloma and the FGFR3 mutation is a K650E mutation.

In some embodiments, the disease or disorder characterized by mutations leading to increased activity of FGFR3 is multiple myeloma or severe achondroplasia with developmental delay and acanthosis nigricans (SADDAN) and the FGFR3 mutation is a K650M mutation.

In some embodiments, the disease or disorder characterized by mutations leading to increased activity of FGFR3 is bladder cancer and the FGFR3 mutation is a S294C/Y375C mutation.

Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject in need thereof one or more second therapeutic agents. The choice of second therapeutic agent may be made from any second therapeutic agent known to be useful for co-administration with meclizine. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula I, Ia or Ib, or a pharmaceutically acceptable salt or hydrate thereof, and a nicotine patch for the cessation of smoking.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I, Ia, or Ib alone or together with one or more of the above-described second therapeutic agents in the manufacture of a medicament, either as a single composition or as separate dosage forms, for treatment in a subject of a disease, disorder or symptom set forth above. Another aspect of the invention is a compound of Formula I, Ia or Ib for use in the treatment in a subject of a disease, disorder or symptom thereof delineated herein.

### EXAMPLES

### Example 1

### 1-((4Chlorophenyl)(phenyl)methyl)-4-((3-(methyl-d₃)phenyl)methyl-d₂)piperazine, di-hydrochloride salt (Compound 167)

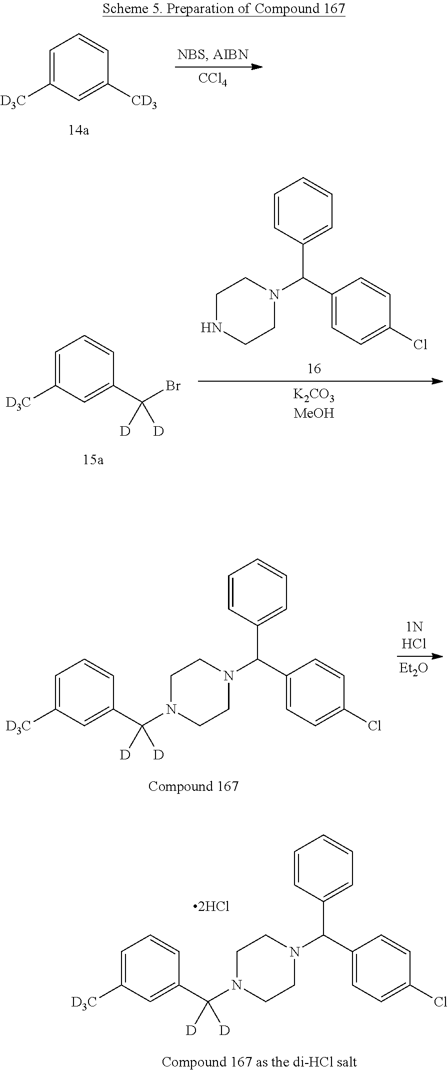

Step 1. 1-(Bromomethyl-d₂)-3-(methyl-d₃)benzene (15a). A mixture of m-xylene-d₆ (CDN, 99 atom % D, 0.5 g, 4.47 mmol, 1 equiv), N-bromosuccinimide (0.79 g, 4.47 mmol, 1 equiv) and azobisisobutyronitrile (AIBN) (0.15 g, 0.91 mmol, 0.2 equiv) in CC1₄ (25 mL) was stirred at reflux for 6.5 hours. AIBN (0.15 g, 0.91 mmol, 0.2 equiv) was added again at 1, 3 and 5 hours. The reaction mixture was cooled to room temperature after 7 hours, filtered and concentrated under reduced pressure to give 15a (1.7 g, >99% yield) as a pale yellow oil which was used without further purification.

Step 2. 1-((4-Chlorophenyl)(phenyl)methyl)-4-((3-(methyl-d₃)phenyl)methyl-d₂)piperazine, di-hydrochloride salt (Compound 167). A mixture of 15a (1.7 g, 4.47 mmol, 1 equiv), commercially available 16 (1.3 g, 4.6 mmol, 1.03 equiv), and potassium carbonate (0.64 g, 4.6 mmol, 1.02 equiv) in methanol (50 mL) was stirred for 16 hours. The reaction mixture was concentrated under reduced pressure. The residue was treated with a saturated sodium bicarbonate solution (100 mL) and extracted three times with ethyl acetate (3×75 mL). The combined organic layers were dried over sodium sulfate, filtered, and concentrated under reduced pressure. The residue was purified using an AnaLogix automated chromatography system (80 g silica gel column), eluting with a gradient of 0-50% ethyl acetate/hexanes, to give Compound 167 (0.51 g, 29% yield) as a pale yellow oil. The oil was re-dissolved in diethyl ether (30 mL) and treated with a 1N HCl solution in ether (10 mL) to give a white suspension. Filtration, washing the solid with diethyl ether, and drying under reduced pressure at 40° C. afforded Compound 167 di-HCl salt (560 mg, 27% overall yield) as a white solid. ¹H-NMR (CD₃OD, 400 MHz) δ 7.60-7.64 (m, 4H), 7.31-7.42 (m, 9H), 4.85 (s, 1H), 3.51 (br s, 4H), 3.32-3.34 (br s, 4H); LCMS m/z=396, 398 [M+H]⁺; Elemental Analysis based on C₂₅H₂₄D₅Cl₃N₂: calculated C=64.45, H=6.23, N=5.97, Cl=22.68, found C=64.27, H=6.30, N=5.81, Cl=21.19.

### Example 2

### 1-(94-Chlorophenyl)(phenyl)methyl)-4-((3-methylphenyl)methyl-d₂)piperazine, di-hydrochloride Salt (Compound 103)

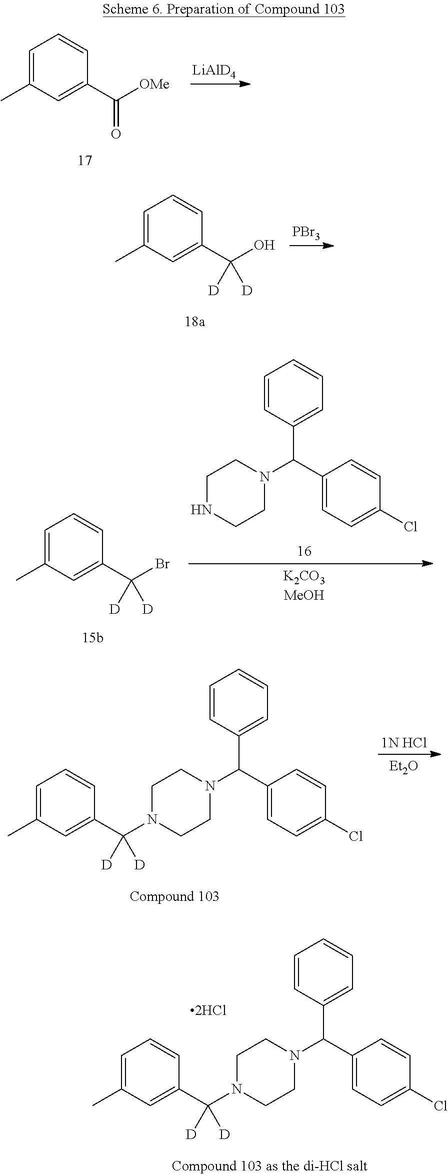

Step 1. 3-Methylphenylmethan-d₂-ol (18a). A suspension of lithium aluminum deuteride (BOC Sciences, 98 atom % D, 0.21 g, 5 mmol, 4 equiv) in tetrahydrofuran (10 mL) was stirred at 0-5° C. under nitrogen. A solution of commercially available 17 (0.75 g, 5 mmol, 1 equiv) in tetrahydrofuran (2 mL) was added dropwise over 5 minutes. The reaction mixture was warmed to room temperature over 21 hours. Deuterium oxide (Aldrich, 99 atom % D, 0.3 mL), a 50% sodium deuteroxide solution in deuterium oxide (Aldrich, 99 atom % D, 0.3 mL) and deuterium oxide (1.0 mL) were added sequentially over 15 minutes. The reaction mixture was filtered, dried over sodium sulfate, filtered again and concentrated under reduced pressure to give 18a (0.46 g, 74% yield) as a clear, colorless oil.

Step 2. 1-(Bromomethyl-d₂)-3-methylbenzene (15b). A solution of 18a (0.46 g, 3.7 mmol, 1.0 equiv) in diethyl ether (6 mL) was stirred at −20° C. A solution of phosphorus tribromide (0.50 g, 0.17 mL, 1.9 mmol, 1.5 equiv) in hexanes (1 mL) was added dropwise over 5 minutes. The reaction mixture was stirred at −20° C. to −10° C. for 20 minutes, then warmed to room temperature over 60 minutes. The organic solution was decanted from a yellow viscous oil, diluted with diethyl ether (10 mL), washed with ice-cold saturated sodium bicarbonate solution (5 mL), dried over sodium sulfate, filtered and concentrated under reduced pressure to give 15b (0.5 g, 72% yield) as a pale yellow oil.

Step 3. 1-((4-Chlorophenyl)(phenyl)methyl)-4-((3-methylphenyl)methyl-d₂)piperazine, di-hydrochloride salt (Compound 103). A mixture of 15b (0.5 g, 3.7 mmol, 1.0 equiv), commercially available 16 (1.1 g, 3.7 mmol, 1.0 equiv) and potassium carbonate (0.52 g, 3.7 mmol, 1.0 equiv) in methanol (40 mL) was stirred for 22 hours at room temperature. The reaction mixture was concentrated under reduced pressure. The residue was treated with a saturated sodium bicarbonate solution (100 mL) and extracted with ethyl acetate (3×75 mL). The combined organic layers were dried over sodium sulfate, filtered and concentrated under reduced pressure. The residue was purified using an AnaLogix automated chromatography system (80 g silica gel column), eluting with a gradient of 0-50% ethyl acetate/hexanes, to give Compound 103 (0.44 g, 30% yield) as a pale yellow oil. The oil was re-dissolved in diethyl ether (30 mL) and treated with a 1N HCl solution in ether (10 mL) to give a white suspension. Filtration, washing the solid with ether, and drying under reduced pressure at 40° C. afforded Compound 103 di-HCl salt (440 mg, 25% overall yield) as a white solid. ¹H-NMIR (CD₃OD, 400 MHz) δ 7.59-7.63 (m, 4H), 7.31-7.42 (m, 9H), 4.85 (s, 1H), 3.50-3.52 (br s, 4H), 3.32-3.34 (br s, 4H); LCMS m/z=393, 395 [M+H]⁺; Elemental Analysis based on C₂₅H₂₇D₂Cl₃N₂: calculated C=64.45, H=6.27, N=6.01, Cl=22.83, found C=64.87, H=6.27, N=6.02, Cl=21.96.

### Example 3

### 1-((4-Chlorophenyl)(phenyl)methyl)-4-(3-(methyl-d₃)benzyl)piperazine, di-hydrochloride Salt (Compound 162)

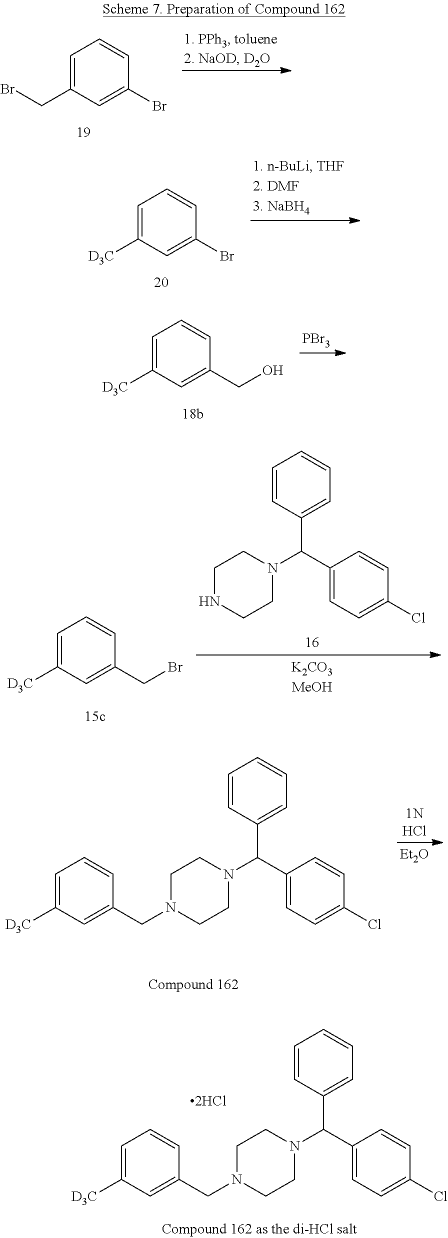

Step 1. 1-Bromo-3-(methyl-d₃)benzene (20). A mixture of commercially available 3-bromobenzylbromide (10.0 g, 40 mmol, 1.0 equiv) and triphenylphosphine (10.5 g, 40 mmol, 1.0 equiv) was stirred in toluene (120 mL) heated to reflux under nitrogen for 8 hours. The white suspension was cooled to room temperature, filtered and triturated with hexanes (100 mL), and filtered again. The white solid was dried under reduced pressure. This phosphonium salt was stirred vigorously in a mixture of tetrahydrofuran (80 mL), deuterium oxide (Aldrich, >99 atom % D, 40 mL) and a 40% sodium deuteroxide in deuterium oxide solution (50 g, Aldrich, 99 atom % D) for 16 hours. The pale yellow biphasic mixture was extracted with diethyl ether (3×75 mL). The combined organic layers were dried over sodium sulfate, filtered, and concentrated under reduced pressure (˜60 torr). The residue was triturated with hexanes (100 mL) and filtered. The filtrate was concentrated under reduced pressure (˜60 Torr) to give 20 (4.44 g, 64% yield) as a pale yellow oil.

Step 2. (3-(Methyl-d₃)phenyl)methanol (18b). A 2.5 M n-butyl lithium solution in hexanes (5.7 mL, 14.1 mmol, 1.0 equiv) was added to a solution of 20 (2.5 g, 14.1 mmol, 1.0 equiv) in tetrahydrofuran (25 mL) at −78° C. under nitrogen over 20 minutes. Stirring was continued at −78° C. for 30 minutes. N,N-Dimethylformamide (1.0 g, 1.1 mL, 14.1 mmol, 1.0 equiv) was added in one portion and the reaction mixture was warmed to room temperature over 2 hours. The reaction mixture was poured into deuterium oxide (50 g, Aldrich, >99 atom % D). The mixture was extracted with diethyl ether (3×50 mL), dried over sodium sulfate, filtered and concentrated under reduced pressure to give the crude aldehyde as a pale yellow oil. The oil was stirred in methanol (25 mL) at 0-5° C. Sodium borohydride (1.29 g, 32 mmol, 9.1 equiv) was added portionwise over 15 minutes. The reaction mixture was stirred at 0-5° C. for 60 minutes and then mixed with diethyl ether (30 mL) and water (30 mL). The layers were separated. The aqueous layer was extracted with diethyl ether (2×30 mL). The combined organic extracts were dried over sodium sulfate, filtered, and concentrated under reduced pressure to give 18b (643 mg, 36% yield) as a pale yellow oil.

Step 3. 1-(Bromomethyl)-3-(methyl-d₃)benzene (15c). A solution of 18b (643 mg, 5.1 mmol, 1.0 equiv) in ether (6 mL) was stirred at −20° C. A solution of phosphorus tribromide (0.23 g, 2.6 mmol, 1.5 equiv) in hexanes (1 mL) was added dropwise over 5 minutes. The reaction mixture was stirred at −20° C. to −10° C. for 20 minutes, then warmed to room temperature over 60 minutes. The organic solution was decanted from a yellow viscous oil, diluted with diethyl ether (10 mL), washed with ice-cold saturated sodium bicarbonate solution (5 mL), dried over sodium sulfate, filtered, and concentrated under reduced pressure to give 15c (967 mg, 100% yield) as a pale yellow oil.

Step 4. 1-((4-Chlorophenyl)(phenyl)methyl)-4-(3-(methyl-d₃)benzyl)piperazine, di-hydrochloride salt (Compound 162). A mixture of 15c (0.97 g, 5.1 mmol, 1.0 equiv), commercially available 16 (1.5 g, 5.1 mmol, 1.0 equiv) and potassium carbonate (0.71 g, 5.1 mmol, 1.0 equiv) in methanol (40 mL) was stirred for 24 hours at room temperature. The solvent was concentrated under reduced pressure. The residue was treated with a saturated sodium bicarbonate solution (100 mL) and extracted with ethyl acetate (3×75 mL). The combined organic layers were dried over sodium sulfate, filtered, and concentrated under reduced pressure. The residue was purified using an AnaLogix automated chromatography system (40 g silica gel column), eluting with a gradient of 0-50% ethyl acetate/hexanes, to give Compound 162 (0.79 g, 39% yield) as a pale yellow oil. The oil was re-dissolved in diethyl ether (30 mL) and treated with a IN HCl solution in ether (10 mL) to give a white suspension. Filtration, washing the solid with diethyl ether and drying under reduced pressure at 40° C. afforded Compound 162 di-HCl salt (0.80 g, 33% overall yield) as a white solid. ¹H-NMR (CD₃OD, 400 MHz) δ 7.58-7.62 (m, 4H), 7.30-7.41 (m, 9H), 4.85 (s, 1H), 4.37 (s, 2H), 3.50 (br s, 4H), 3.32-3.34 (br s, 4H); LCMS m/z=394, 396 [M+H]⁺.

### Example 4

### Preparation of R and S Enantiomers (General Methods)

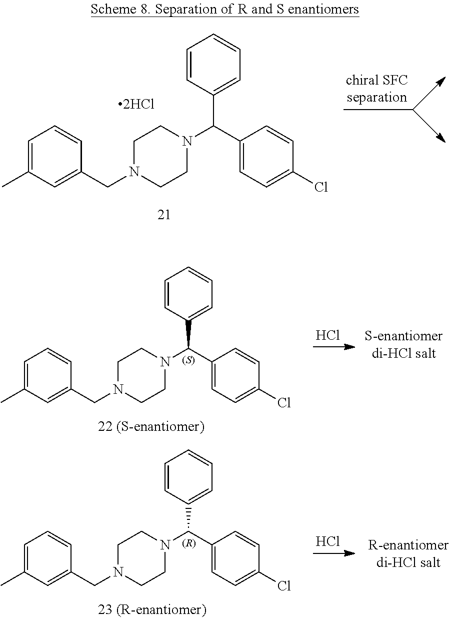

Step 1. Chiral SFC separation (General Method A). Commercially available racemic 21 (3.02 g) was dissolved in 250 mL of 80:20 isopropyl alcohol (IPA)/acetonitrile, for a concentration of 0.012 g/mL. Via repeated injections of 4.50 mL each, the material was separated via chiral SFC (supercritical fluid chromatography) under the following separatory conditions:

Column type: RegisPack; Dimensions: L 250 mm, ID 30 mm, particle size 5 μm; Mobile phase: 40% IPA (isopropyl alcohol)+0.5% diethylamine/60% CO_{2;} Detection wavelength: 220 nm; Flow rate: 80.00 g/min; Co-solvent flow rate: 32.00 mL/min.

Fractions were collected and solvent was removed under reduced pressure at 40° C. to afford Isomer 1 (1.11 g brown oil) and Isomer 2 (1.91 g brown oil), each with residual mobile phase present. Chiral purity of each isomer was assessed as 100% ee via chiral HPLC under the following analytical conditions:

Column type: RegisPack; Dimensions: L 250 mm, ID 4.6 mm, particle size 5 μm; Mobile phase: 85% Hexane/15% IPA +0.1% diethylamine; Flow rate: 1.50 mL/min; Pressure: 67 bar; Rₜ of Isomer 1: 2.337 min; Rₜ of Isomer 2: 2.816 min.

Step 2a. Preparation of a Single-Enantiomer di-HCl Salt (General Method B).

Isomer 1 oil was dissolved in dichloromethane with several drops of methanol and purified using an AnaLogix automated chromatography system (25 g column), eluting with a gradient of 0 to 10% methanol in dichloromethane over 20 minutes. Pure fractions were concentrated under reduced pressure to give 860 mg of a colorless film. The residue was dissolved in isopropanol (30 mL) and 4N HCl in dioxane (3 mL) was added. The mixture was cooled to 0° C. and stirred for 30 minutes. The resulting white solid was filtered, washed with isopropanol and dried in a vacuum oven overnight at 40° C. to give 550 mg of a white solid. Elemental analysis indicated this material was the di-hydrochloride salt. Additional material of lesser chemical purity was present in the filtrate and was not isolated.

Step 2b. Preparation of a Single-Enantiomer di-HCl Salt (General Method C).

Isomer 2 oil was dissolved in dichloromethane with several drops of methanol and purified using an AnaLogix automated chromatography system (25 g column), eluting with a gradient of 0 to 7% methanol in dichloromethane over 30 minutes. Pure fractions were concentrated under reduced pressure to give 1.1 g of a colorless film. The residue was dissolved in dioxane (5 mL) and 4N HCl in dioxane (0.7 mL) was added, forming a thick suspension. Diethyl ether (15 mL) was added and the mixture was stirred for 30 minutes. The resulting white solid was filtered, washed with dioxane and dried in a vacuum oven for 72 hours at 40° C. to give 950 mg of a white solid which contained significant residual dioxane. The solids were triturated with isopropanol (40 mL), filtered and dried in a vacuum oven for 4 hours at 40° C. to give 320 mg of a white solid. Elemental analysis indicated this material was the di-hydrochloride salt.

Additional Isomer 2 material was present in the filtrate, which was concentrated under reduced pressure. The residue was dissolved in dichloromethane, and the solution was filtered and concentrated. The residue was dried in a vacuum oven to give 560 mg of a beige solid. Elemental analysis indicated this material was the mono-hydrochloride salt. The mono-hydrochloride salt was dissolved in isopropanol (25 mL) and 4N HCl in dioxane (5 mL) was added. The mixture was cooled to 0° C., stirred for 30 minutes, filtered, washed with isopropanol and dried in a vacuum oven at 40° C. overnight to give 220 mg of a white solid. Elemental analysis indicated this material was the di-hydrochloride salt.

The filtrate from above was concentrated under reduced pressure and dried in a vacuum oven overnight at 40° C. to give an additional crop of 320 mg as a beige solid. Elemental analysis indicated this material was the di-hydrochloride salt.

Step 3. Identification of R and S enantiomers. Isomer 1 and Isomer 2 were compared to a known sample of the R enantiomer via chiral HPLC under the following analytical conditions. Isomer 1 was identified as the S enantiomer (22) and Isomer 2 was identified as the R enantiomer (23).

Column type: RegisPack; Dimensions: L 250 mm, ID 4.6 mm, particle size 5 μm; Mobile phase: 95% Hexane/5% IPA+0.1% diethylamine; Flow rate: 0.7 mL/min; Rₜ of S enantiomer: 5.785 min; Rₜ of R enantiomer: 7.713 min.

### Example 5

### Preparation of R and S enantiomers of Compounds 167, 103, and 162

Racemic Compound 167 is separated via General Method A to afford the R and S enantiomers. Purification and salting via General Method B affords the di-HCl salts of the R and S enantiomers of Compound 167. The enantiomers are identified via chiral HPLC comparison with a known sample of the protio R enantiomer using the provided analytical conditions.

Racemic Compound 103 is separated via General Method A to afford the R and S enantiomers. Purification and salting via General Method B affords the di-HCl salts of the R and S enantiomers of Compound 103. The enantiomers are identified via chiral HPLC comparison with a known sample of the protio R enantiomer using the provided analytical conditions.

Racemic Compound 162 is separated via General Method A to afford the R and S enantiomers. Purification and salting via General Method B affords the di-HCl salts of the R and S enantiomers of Compound 162. The enantiomers are identified via chiral HPLC comparison with a known sample of the protio R enantiomer using the provided analytical conditions.

### Example 6

### Evaluation of Metabolic Stability

Microsomal Assay: Human liver microsomes (20 mg/mL) are obtained from Xenotech, LLC (Lenexa, KS). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I, Ia or Ib and the positive control, 7-ethoxycoumarin (1 μM). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining (ln) vs incubation time]

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 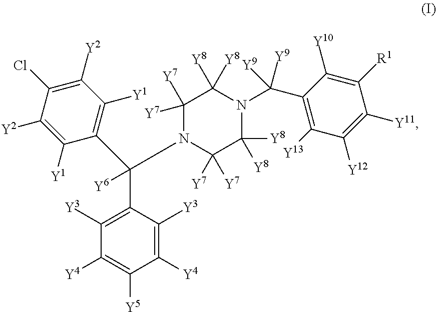
or a pharmaceutically acceptable salt or hydrate thereof, wherein:
each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium;
R¹ is —CH₃, —CH₂D, —CHD₂, or —CD₃;
when each Y⁷ is hydrogen, then R¹ is —CH₂D, —CHD₂, or —CD₃; and
when each Y⁷ and each Y⁸ is deuterium and R¹ is —CH₃, at least one of Y¹, Y², Y³, Y⁴, Y⁵, Y⁶, Y⁹, Y¹⁰, Y¹¹, Y¹² or Y¹³ is deuterium.

2-3. (canceled)

4. The compound of claim 1, wherein each Y⁷ is the same and each Y⁸ is the same.

5-6. (canceled)

7. The compound of claim 1, wherein R¹ is —CH₃ or —CD₃.

8. The compound of claim 1, wherein:
each Y¹ and Y² are all the same;
each Y³, each Y⁴ and Y⁵ are all the same;
each Y⁹ is the same;
Y¹⁰, Y¹¹, Y¹² and Y¹³ are the same; and
R¹ is selected from —CH₃ and —CD₃.

9. The compound of claim 8, wherein each Y⁷ is the same; and each Y⁸ is the same.

10. The compound of claim 9, wherein each Y⁷ and each Y⁸ are all the same.

11. The compound of claim 1, wherein any atom not designated as deuterium is present at its natural isotopic abundance.

12. The compound of claim 1, wherein each Y¹ and each Y² are all the same; each Y³, each Y⁴ and Y⁵ are all the same; each Y⁷ and each Y⁸ are all the same; Y¹⁰,Y¹¹, Y¹² and Y¹³ are the same; and the compound is selected from any one of the compounds set forth in the table below:

| Compound | | | | | | Y¹⁰/Y¹¹/ | |
|---|---|---|---|---|---|---|---|
| # | Y¹/Y² | Y³/Y⁴/Y⁵ | Y⁶ | Y⁷/Y⁸ | Y⁹ | Y¹²/Y¹³ | R¹ |
| 100 | D | H | H | H | H | H | CH₃ |
| 101 | H | D | H | H | H | H | CH₃ |
| 102 | H | H | D | H | H | H | CH₃ |
| 103 | H | H | H | H | D | H | CH₃ |
| 104 | H | H | H | H | H | D | CH₃ |
| 105 | D | D | H | H | H | H | CH₃ |
| 106 | D | H | D | H | H | H | CH₃ |
| 107 | D | H | H | D | H | H | CH₃ |
| 108 | D | H | H | H | D | H | CH₃ |
| 109 | D | H | H | H | H | D | CH₃ |
| 110 | H | D | D | H | H | H | CH₃ |
| 111 | H | D | H | D | H | H | CH₃ |
| 112 | H | D | H | H | D | H | CH₃ |
| 113 | H | D | H | H | H | D | CH₃ |
| 114 | H | H | D | D | H | H | CH₃ |
| 115 | H | H | D | H | D | H | CH₃ |
| 116 | H | H | D | H | H | D | CH₃ |
| 117 | H | H | H | D | D | H | CH₃ |
| 118 | H | H | H | D | H | D | CH₃ |
| 119 | H | H | H | H | D | D | CH₃ |
| 120 | D | D | D | H | H | H | CH₃ |
| 121 | D | D | H | D | H | H | CH₃ |
| 122 | D | D | H | H | D | H | CH₃ |
| 123 | D | D | H | H | H | D | CH₃ |
| 124 | D | H | D | D | H | H | CH₃ |
| 125 | D | H | D | H | D | H | CH₃ |
| 126 | D | H | D | H | H | D | CH₃ |
| 127 | D | H | H | D | D | H | CH₃ |
| 128 | D | H | H | D | H | D | CH₃ |
| 129 | D | H | H | H | D | D | CH₃ |
| 130 | H | D | D | D | H | H | CH₃ |
| 131 | H | D | D | H | D | H | CH₃ |
| 132 | H | D | D | H | H | D | CH₃ |
| 133 | H | D | H | D | D | H | CH₃ |
| 134 | H | D | H | D | H | D | CH₃ |
| 135 | H | D | H | H | D | D | CH₃ |
| 136 | H | H | D | D | D | H | CH₃ |
| 137 | H | H | D | D | H | D | CH₃ |
| 138 | H | H | D | H | D | D | CH₃ |
| 139 | H | H | H | D | D | D | CH₃ |
| 140 | D | D | D | D | H | H | CH₃ |
| 141 | D | D | D | H | D | H | CH₃ |
| 142 | D | D | D | H | H | D | CH₃ |
| 143 | D | D | H | D | D | H | CH₃ |
| 144 | D | D | H | D | H | D | CH₃ |
| 145 | D | D | H | H | D | D | CH₃ |
| 146 | D | H | D | D | D | H | CH₃ |
| 147 | D | H | D | D | H | D | CH₃ |
| 148 | D | H | D | H | D | D | CH₃ |
| 149 | D | H | H | D | D | D | CH₃ |
| 150 | H | D | D | D | D | H | CH₃ |
| 151 | H | D | D | D | H | D | CH₃ |
| 152 | H | D | D | H | D | D | CH₃ |
| 153 | H | D | H | D | D | D | CH₃ |
| 154 | H | H | D | D | D | D | CH₃ |
| 155 | D | D | D | D | D | H | CH₃ |
| 156 | D | D | D | D | H | D | CH₃ |
| 157 | D | D | D | H | D | D | CH₃ |
| 158 | D | D | H | D | D | D | CH₃ |
| 159 | D | H | D | D | D | D | CH₃ |
| 160 | H | D | D | D | D | D | CH₃ |
| 161 | D | D | D | D | D | D | CH₃ |
| 162 | H | H | H | H | H | H | CD₃ |
| 163 | D | H | H | H | H | H | CD₃ |
| 164 | H | D | H | H | H | H | CD₃ |
| 165 | H | H | D | H | H | H | CD₃ |
| 166 | H | H | H | D | H | H | CD₃ |
| 167 | H | H | H | H | D | H | CD₃ |
| 168 | H | H | H | H | H | D | CD₃ |
| 169 | D | D | H | H | H | H | CD₃ |
| 170 | D | H | D | H | H | H | CD₃ |
| 171 | D | H | H | D | H | H | CD₃ |
| 172 | D | H | H | H | D | H | CD₃ |
| 173 | D | H | H | H | H | D | CD₃ |
| 174 | H | D | D | H | H | H | CD₃ |
| 175 | H | D | H | D | H | H | CD₃ |
| 176 | H | D | H | H | D | H | CD₃ |
| 177 | H | D | H | H | H | D | CD₃ |
| 178 | H | H | D | D | H | H | CD₃ |
| 179 | H | H | D | H | D | H | CD₃ |
| 180 | H | H | D | H | H | D | CD₃ |
| 181 | H | H | H | D | D | H | CD₃ |
| 182 | H | H | H | D | H | D | CD₃ |
| 183 | H | H | H | H | D | D | CD₃ |
| 184 | D | D | D | H | H | H | CD₃ |
| 185 | D | D | H | D | H | H | CD₃ |
| 186 | D | D | H | H | D | H | CD₃ |
| 187 | D | D | H | H | H | D | CD₃ |
| 188 | D | H | D | D | H | H | CD₃ |
| 189 | D | H | D | H | D | H | CD₃ |
| 190 | D | H | D | H | H | D | CD₃ |
| 191 | D | H | H | D | D | H | CD₃ |
| 192 | D | H | H | D | H | D | CD₃ |
| 193 | D | H | H | H | D | D | CD₃ |
| 194 | H | D | D | D | H | H | CD₃ |
| 195 | H | D | D | H | D | H | CD₃ |
| 196 | H | D | D | H | H | D | CD₃ |
| 197 | H | D | H | D | D | H | CD₃ |
| 198 | H | D | H | D | H | D | CD₃ |
| 199 | H | D | H | H | D | D | CD₃ |
| 200 | H | H | D | D | D | H | CD₃ |
| 201 | H | H | D | D | H | D | CD₃ |
| 202 | H | H | D | H | D | D | CD₃ |
| 203 | H | H | H | D | D | D | CD₃ |
| 204 | D | D | D | D | H | H | CD₃ |
| 205 | D | D | D | H | D | H | CD₃ |
| 206 | D | D | D | H | H | D | CD₃ |
| 207 | D | D | H | D | D | H | CD₃ |
| 208 | D | D | H | D | H | D | CD₃ |
| 209 | D | D | H | H | D | D | CD₃ |
| 210 | D | H | D | D | D | H | CD₃ |
| 211 | D | H | D | D | H | D | CD₃ |
| 212 | D | H | D | H | D | D | CD₃ |
| 213 | D | H | H | D | D | D | CD₃ |
| 214 | H | D | D | D | D | H | CD₃ |
| 215 | H | D | D | D | H | D | CD₃ |
| 216 | H | D | D | H | D | D | CD₃ |
| 217 | H | D | H | D | D | D | CD₃ |
| 218 | H | H | D | D | D | D | CD₃ |
| 219 | D | D | D | D | D | H | CD₃ |
| 220 | D | D | D | D | H | D | CD₃ |
| 221 | D | D | D | H | D | D | CD₃ |
| 222 | D | D | H | D | D | D | CD₃ |
| 223 | D | H | D | D | D | D | CD₃ |
| 224 | H | D | D | D | D | D | CD₃ |
| 225 | D | D | D | D | D | D | CD₃ |

wherein any atom not designated as deuterium is present at its natural isotopic abundance.

13. A pharmaceutical composition comprising a compound of Formula I: 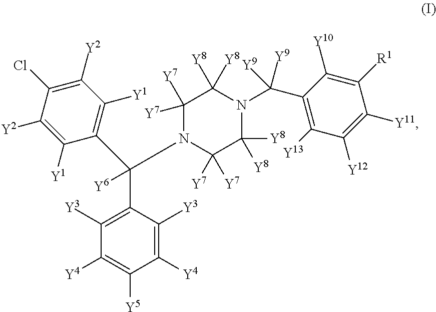
or a pharmaceutically acceptable salt or hydrate thereof, wherein:
each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium;
R¹ is —CH₃, —CH₂D, —CHD₂, or —CD₃; and
when each Y is hydrogen, then R¹ is —CH₂D, —CHD₂, or —CD₃; and a pharmaceutically acceptable carrier.

14. (canceled)

15. A method of agonizing the activity of the Constitutive Androstane Receptor in a cell, comprising contacting the cell with a compound of Formula I: 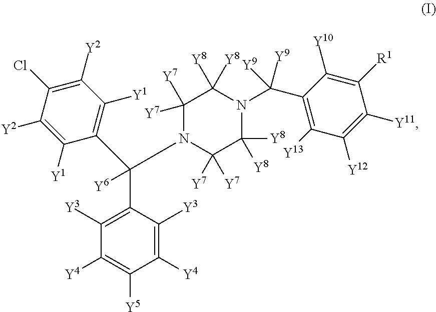
or a pharmaceutically acceptable salt or hydrate thereof, wherein:
each instance of Y¹, Y², Y³, Y⁴, Y⁵, Y⁶, Y⁷, Y⁸, Y⁹, Y¹⁰, Y¹¹, Y¹² and Y¹³ is independently selected from hydrogen and deuterium;
R¹ is —CH₃, —CH₂D, —CHD₂, or —CD₃; and
when each Y is hydrogen, then R¹ is —CH₂D, —CHD₂, or—CD₃.

16.-18. (canceled)

19. A method of treating a subject suffering from or susceptible to a disease or disorder selected from Huntington's disease and other polyQ disorders; ischemia-perfusion injury; heart attack; stroke and other diseases involving oxidative damage; achondroplasia, cartilage hypoplasia, Tana Tofo Rick bone dysplasia, Crouzon's disease, distal middle limb dysplasia, Mu severe cartilage with developmental delay, acanthosis nigricans and other systemic bone diseases characterized by over-activation of FGFR3; smoking/nicotine addiction, and vertigo, comprising the step of administering to the subject in need thereof a compound of Formula (I) of claim 1.

20. The method of claim 19, wherein the disease or disorder is selected from smoking/nicotine addiction and vertigo.

21. The method of claim 19, comprising the additional step of administering to the subject in need thereof a second therapeutic agent selected from an agent useful in the treatment of a disease or condition selected from smoking addiction (e.g., useful in aiding in smoking cessation); vertigo; motion sickness; systemic bone disease; arthritis; nausea; vomiting; neurodegenerative disorders such as ALS, ataxia, Friedrich's dementia, Alzheimer's disease, Parkinson's disease, Huntington's disease; ischemic disorders such as myocardial ischemia, renal ischemia and stroke; or a second therapeutic agent that has side effects which are attenuated or eliminated by the compound of Formula (I).

22. The method of claim 21, wherein the second agent is selected from an anti-cancer agent, an oral contraceptive, and other agents known to cause nausea and/or vomiting.

23. The method of claim 21, wherein the second agent is a nicotine patch.

24. A method of treating a subject suffering from or susceptible to a disease or disorder characterized by mutations leading to increased activity of FGFR3, comprising the step of administering to the subject in need thereof a compound of claim 1.

25. The method of claim 24, wherein the disease or disorder characterized by mutations leading to increased activity of FGFR3 is a cancer.

26. The method of claim 25, wherein the cancer is selected from multiple myeloma, bladder cancer, prostate cancer, rhabdomycosarcoma, non-small cell lung cancer (NSCLC), oral squamous cell carcinoma, and thanatophoric dysplasia type II.

27. The method of claim 24, comprising the additional step of administering to the subject in need thereof a second therapeutic agent selected from an agent useful in the treatment of a cancer selected from multiple myeloma, bladder cancer, prostate cancer, rhabdomycosarcoma, non-small cell lung cancer (NSCLC), oral squamous cell carcinoma, and thanatophoric dysplasia type II.

28. The compound of claim 1, wherein the deuterium incorporation at each designated deuterium atom is at least 90%.

29. The compound of claim 1, wherein the deuterium incorporation at each designated deuterium atom is at least 95%.
